(19) United States
(12) Patent Application Publication
(10) Pub. No.: US 2010/0055167 A1
(43) Pub. Date: Mar. 4, 2010

(54) STEM CELL DELIVERY OF ANTI-NEOPLASTIC MEDICINE

(76) Inventors: Alex Zhang, Beijing (CN); Yunqian Guan, Beijing (CN); Ling Chen, Beijing (CN)

Correspondence Address: FOLEY & LARDNER LLP, 150 EAST GILMAN STREET, P.O. BOX 1497, MADISON, WI, 53701-1497, US

(21) Appl. No.: 12/546,969

(22) Filed: Aug. 25, 2009

(30) Foreign Application Priority Data
Aug. 29, 2008 (CN) 200810146798.5

(51) Int. Cl. A61K 9/127; A61K 48/00; A61K 39/395; A61P 35/00; A61K 9/14

(52) U.S. Cl. 424/450; 424/93.21; 424/178.1; 424/489; 977/773

(57) ABSTRACT

The present disclosure provides a modified stem cell comprising a stem cell and at least one controlled-release vehicle, wherein the at least one controlled-release vehicle includes at least one anti-neoplastic agent and at least one targeting moiety, and wherein the modified stem cell is characterized by an ability to target one or more glioma cells. The disclosure also provides pharmaceutical compositions and methods of treating a glioma by administering a therapeutically effective amount of the modified stem cells.

### CROSS-REFERENCE TO RELATED APPLICATIONS

The present application claims priority to Chinese Patent Application No. 200810146798.5, filed Aug. 29, 2008, the entire contents of which are incorporated by reference in its entirety.

### BACKGROUND

The present technology is in the field of medical treatment with stem cells. More particularly, the technology relates to treatments for central nervous system (CNS) tumors.

### SUMMARY

In one aspect, the disclosure provides a modified stem cell comprising a stem cell and at least one controlled-release vehicle, wherein the at least one controlled-release vehicle includes at least one anti-neoplastic agent and at least one targeting moiety, and wherein the modified stem cell is characterized by an ability to target one or more glioma cells. In one embodiment, the at least one controlled-release vehicle is selected from the group consisting of: nanoparticles; biocompatible polymers; polymeric matrices; liposomes; and lipospheres.

In one embodiment, the at least one controlled-release vehicle comprises at least one nanoparticle. In another embodiment, the nanoparticle has a diameter of about 10 to about 600 nm. In another embodiment, the nanoparticle has a diameter of about 200 to about 400 nm. In one embodiment, the nanoparticle is a silicate nanoshell. In one embodiment, the silicate nanoshell is loaded with the at least one anti-neoplastic agent. In one embodiment, the controlled-release vehicle has a controlled release rate. In another embodiment, the controlled release rate is from about 5 days to about 31 days.

In one embodiment, the stem cell is selected from the group consisting of: mesenchymal stem cells; neural stem cells; and embryonic stem cells. In one embodiment, the at least one targeting moiety is conjugated to the surface of the controlled-release vehicle. In one embodiment, the at least one targeting moiety is an antibody, or fragment thereof. In another embodiment, the antibody is a monoclonal antibody or a polyclonal antibody, or fragment thereof. In one embodiment, the at least one targeting moiety specifically binds a surface antigen on the stem cell. In one embodiment, the surface antigen is selected from the group consisting of: CD105 (SH2); CD73 (SH3/4); CD44; CD90 (Thy-1); CD71; Stro-1; CD106; and CD166. In another embodiment, the surface antigen is CD90.

In some embodiments, the at least one anti-neoplastic agent is selected from the group consisting of: a chemotherapeutic agent; a protein-based pharmaceutical; and a nucleic acid-based pharmaceutical. In some embodiments, the at least one anti-neoplastic agent is selected from the group consisting of: asparaginase; adriamycin; alkaloids; alkylating agent; altretamine; amsacrine; anti-metabolite compound; antitumour antibiotics; azathioprine; bleomycin sulfate; busulfan; camptothecins; carboplatin; carmustine; chlorambucil; cisplatin; cladribine; cyclophosphamide; Cytarabine; Dacarbazine; dactinomycin; daunorubicin; docetaxel; doxorubicin hydrochloride; epipodophyllotoxins; epirubicin hydrochloride; estramustine sodium phosphate; etoposide; etoposide phosphate; finasteride; fludarabine phosphate; fluorouracil; gonadotropin-releasing hormone agonists (GnRH); Goserelin; hydroxyurea; idarubicin hydrochloride; ifosfamide; irinotecan; lomustine; marimastat; mechlorethamine; mechlorethamine hydrochloride; melphalan; mercaptopurine; methotrexate sodium; mitomycin; mitotane; mitoxantrone hydrochloride; oxaliplatin; paclitaxel; Podophyllotoxin; porfimer sodium; procarbazine hydrochloride; radiotherapeutics; streptozocin; suramin; tamoxifen; taxanes; taxol; teniposide terpenoids; thalidomide; Thioguanine; thiotepa; TNP 470; topoisomerase inhibitors; topotecan; tretinoin (all-trans retinoic acid); vinblastine; vinblastine sulfate; vinca alkaloid; vincristine; vincristine sulfate; Vindesine; vindesine sulfate; and vinorelbine tartrate.

In one embodiment, the nanoparticle further comprises a labeling moiety. In one embodiment, the labeling moiety is fluorescein isothiocyanate (FITC).

In one aspect, the disclosure relates to a pharmaceutical composition comprising the modified stem cell and a pharmaceutically acceptable carrier.

In one aspect, the disclosure relates to a method for the treatment of glioma comprising: administering to a subject in need thereof, a therapeutically effective amount of the modified stem cells. In one embodiment, the administering is by systemic administration. In one embodiment, the administering is by local administration. In one embodiment, the local administration comprises injection into the cranium of the subject.

In one aspect, the disclosure relates to a method for slowing the growth of a glioma or reducing the volume of a glioma tumor comprising: administering to a subject in need thereof, a therapeutically effective amount of the modified stem cells.

In one aspect, the disclosure relates to use of the modified stem cells for the manufacture of a medicament for treating glioma.

In one aspect, the disclosure relates to a kit for the treatment of glioma comprising one or more modified stem cells and instructions for the use of the one or more modified stem cells.

In one aspect, the disclosure relates to a method for making a modified stem cell comprising: contacting a stem cell with at least one controlled-release vehicle having at least one anti-neoplastic agent and at least one targeting moiety. In one embodiment, the stem cell preferentially localizes to a glioma in a subject.

### BRIEF DESCRIPTION OF THE DRAWINGS

FIGS. 1A, 1B, and 1C are micrographs of C6 glioma cells in culture before (FIG. 1A) and after contact (FIGS. 1B, 1C) with adriamycin-loaded, anti-C90-bound silicate nanoparticles.

FIGS. 2A through 2F are micrographs of mesenchymal stem cells contacted with anti-C90, FITC-labeled silicate nanoparticles.

### DETAILED DESCRIPTION

This disclosure is drawn, inter alia, to modified stem cells, as well as related preparation methods, and methods of using the modified stem cells.

In this disclosure, many conventional techniques in molecular biology, protein biochemistry, cell biology, immunology, and microbiology are used. These techniques are well-known and are explained in, e.g., Current Protocols in Molecular Biology, Vols. I-III, Ausubel, Ed. (1997); Sambrook et al, Molecular Cloning: A Laboratory Manual, Second Ed. (Cold Spring Harbor Laboratory Press, Cold Spring Harbor, N.Y., 1989); DNA Cloning: A Practical Approach, Vols. I and II, Glover, Ed. (1985); Oligonuchotide Synthesis, Gait, Ed. (1984); Nucleic Acid Hybridization, Hames & Higgins, Eds. (1985); Transcription and Translation, Hames & Higgins, Eds. (1984); Animal Cell Culture, Freshney, Ed. (1986); Immobilized Cells and Enzymes (IRL Press, 1986); Perbal, A Practical Guide to Molecular Cloning; the series, Meth. Enzymol., (Academic Press, Inc., 1984); Gene Transfer Vectors for Mammalian Cells, Miller & Calos, Eds. (Cold Spring Harbor Laboratory, NY, 1987); and Meth. Enzymol., Vols. 154 and 155, Wu & Grossman, and Wu, Eds., respectively.

In the description that follows, a number of terms are utilized extensively. The explanations are herein provided to facilitate understanding of the technology. The terms provided below are more fully explicated by reference to the specification as a whole.

Unless explicitly described to the contrary, the word “comprise” and variations such as “comprises” or “comprising”, and “having” and/or “including” will be understood to include the information described in the body of the claim, for example, but not to exclude information not explicitly set forth.

The terms “a” and “an” as used herein mean “one or more” unless the singular is expressly specified or the context clearly indicates otherwise.

When referring to a numerical value, the term “about” means plus or minus 10% of the enumerated value, unless otherwise indicated.

As used herein, the “administration” of an agent or drug to a subject or subjects includes any route of introducing or delivering to a subject a compound to perform its intended function. Administration can be carried out by any suitable route, including orally, intranasally, parenterally (intravenously, intramuscularly, intraperitoneally, or subcutaneously), rectally, or topically. Administration includes self-administration and the administration by another.

As used herein, the term “antibody” means a polypeptide comprising a framework region from an immunoglobulin gene or fragments thereof that specifically binds and recognizes an antigen. Use of the term antibody is meant to include whole antibodies, including single-chain whole antibodies, and antibody-related polypeptides. The term “antibody” includes bispecific antibodies and multispecific antibodies so long as they exhibit the desired biological activity or function.

As used herein, an “anti-neoplastic agent” is any compound, composition, admixture, co-mixture or blend which inhibits, eliminates, retards or reverses the neoplastic phenotype of a cell. In some embodiments, the anti-neoplastic agent includes, but is not limited to, a small molecule drug, protein-based pharmaceutical, or nucleic acid-based pharmaceutical.

As used herein, the term “effective amount” or “pharmaceutically effective amount” or “therapeutically effective amount” of a composition, is a quantity sufficient to achieve a desired therapeutic and/or prophylactic effect, e.g., an amount which results in the prevention of, or a decrease in, the symptoms associated with a disease or medical condition that is being treated, e.g., the diseases or medical conditions associated with a target polypeptide. The amount of a composition administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The compositions can also be administered in combination with one or more additional therapeutic compounds.

As used herein, the term “epitope” means any antigenic determinant on an antigen to which the antibody binds. Epitopic determinants usually consist of chemically active surface groupings of molecules such as amino acids or sugar side chains and usually have specific three dimensional structural characteristics, as well as specific charge characteristics. In some embodiments, an epitope is on a stem cell surface antigen and is recognized by a stem cell-specific antibody.

As used herein, the term “immune response” refers to the concerted action of lymphocytes, antigen-presenting cells, phagocytic cells, granulocytes, and/or soluble macromolecules produced by the above cells or the liver (including antibodies, cytokines, and complement) that results in damage to, destruction of, or elimination from the human body of detrimental cells, such as cancerous cells, and metastatic tumor cells.

As used herein, the term “medical condition” includes, but is not limited to, any condition or disease manifested as one or more physical and/or psychological symptoms for which treatment and/or prevention is desirable, and includes previously and newly identified diseases and other disorders. A medical condition may include, but is not limited to, a cancer of the central nervous system, e.g., a glioma.

The term “monoclonal antibody” as used herein refers to an antibody obtained from a population of substantially homogeneous antibodies, i.e., the individual antibodies comprising the population are identical except for possible naturally occurring mutations that may be present in minor amounts. For example, a monoclonal antibody can be an antibody that is derived from a single clone, including any eukaryotic, prokaryotic, or phage clone, and not the method by which it is produced. A monoclonal antibody composition displays a single binding specificity and affinity for a particular epitope. Monoclonal antibodies are highly specific, being directed against a single antigenic site. Furthermore, in contrast to conventional (polyclonal) antibody preparations which may include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. Monoclonal antibodies can be prepared using a wide variety of techniques known in the art including, e.g., but not limited to, hybridoma, recombinant, and phage display technologies. For example, the monoclonal antibodies may be made by the hybridoma method first described by Kohler et al., 1975. Nature 256:495, or may be made by recombinant DNA methods (See, e.g., U.S. Pat. No. 4,816,567). The “monoclonal antibodies” may also be isolated from phage antibody libraries using the techniques described in Clackson et al., 1991. Nature 352:624-628 and Marks et al., 1991. J. Mol. Biol. 222:581-597.

As used herein, the term “nanoparticle” refers to particles having nanoscale dimensions, i.e., having a diameter of from about 1 to about 1000 nanometers, and having any size, shape or morphology. As used herein, the term nanoparticle may include spherical nanoparticles as well as non-spherical nanoparticles. For example, the particles may be elongated as in the case of nanowires, nanotubes, and similar structures. A “nanoshell” is a subspecies of nanoparticles characterized by a discrete core/shell structure in which the shell surrounds at least a portion of a core. The core of the nanoshell may be hollow (i.e., empty or filled with a gas) or it may be filled with a liquid (aqueous, oil, etc.) or a solid different from that of the shell.

As used herein, the terms “peptide,” “polypeptide” and “protein” are used interchangeably, and are understood to mean a molecule comprising two or more amino acids, where the alpha carboxyl group of one is bound to the alpha amino group of another. A peptide may have a C-terminus and an N-terminus, which relate to the carboxy portion of an amino acid on one end of the peptide chain and the amino portion of an amino acid on the other end of the peptide chain.

As used herein, the term “polyclonal antibody” refers to multiple immunoglobulins in antiserum produced to an antigen following immunization, and which may recognize and bind to one or more epitopes to that antigen.

As used herein, the term “small molecule” means a composition that has a molecular weight of less than about 5 kDa. Small molecules can be, e.g., nucleic acids, peptides, polypeptides, glycopeptides, peptidomimetics, carbohydrates, lipids, lipopolysaccharides, combinations of these, or other organic or inorganic molecules.

As used herein, the term “stem cell” generally refers to any cells that have the ability to divide for indefinite periods of time and to give rise to specialized cells. The term “stem cell” includes but is not limited, to the following: a) totipotent cells such as an embryonic stem cell, an extra-embryonic stem cell, a cloned stem cell, a parthenogenesis derived cell, a cell reprogrammed to possess totipotent properties, or a primordial germ cell; b) pluripotent cell such as a hematopoietic stem cell, an adipose derived stem cell, a mesenchymal stem cell, a cord blood stem cell, a placentally derived stem cell, an exfoliated tooth derived stem cells, a hair follicle stem cell or a neural stem cell; and c) a tissue specific progenitor cell such as a precursor cell for the neuronal, hepatic, nephrogenic, adipogenic, osteoblastic, osteoclastic, alveolar, cardiac, intestinal, or endothelial lineage. The cells can be derived, for example, from tissues such as pancreatic tissue, liver tissue, smooth muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, Peyer's patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, and mesentery tissue.

The term “specific binding” refers to that binding which occurs between such paired species as enzyme/substrate, receptor/agonist, antibody/antigen, lectin/carbohydrate, aptamer/ligand, and complementary nucleic acids which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody typically binds to a single epitope and to no other epitope within the family of proteins.

As used herein, the term “subject” means the subject is a mammal, such as a human, but can also be an animal, e.g., domestic animals (e.g., dogs, cats and the like), farm animals (e.g., cows, sheep, pigs, horses and the like) and laboratory animals (e.g., monkey, rats, mice, rabbits, guinea pigs and the like).

As used herein, “targeting moiety” is a molecule that is capable of specific binding with a target. In some embodiments, the targeting moiety is a member of a specific binding pair, e.g., an antigen; ligand; receptor; polyamide; peptide; carbohydrate; oligosaccharide; polysaccharide; low density lipoprotein (LDL) or an apoprotein of LDL; steroid; steroid derivative; hormone; hormone-mimic; lectin; drug; antibiotic; aptamer; DNA; RNA; lipid; or an antibody or antibody-related polypeptide.

As used herein “therapeutic effect” means an effect resulting from treatment of a subject that alters, improves or ameliorates, the symptoms of a disease or condition or that cures a disease or condition.

As used herein, the terms “treating” or “treatment” or “alleviation” refers to both therapeutic treatment and prophylactic or preventative measures, wherein the object is to prevent or slow down (lessen) the targeted pathologic condition or disorder. A subject is successfully “treated” for a disorder if, after receiving a therapeutic amount of the modified stem cells, the subject shows observable and/or measurable reduction in or absence of one or more signs and symptoms of a particular disease or medical condition. For example, for cancer, reduction in the number of cancer cells or absence of the cancer cells; reduction in the tumor size; inhibition (i.e., slow to some extent or stop) of tumor metastasis; inhibition, to some extent, of tumor growth; increase in length of remission, and/or relief to some extent of one or more of the symptoms associated with the cancer; reduced morbidity and mortality, and improvement in quality of life issues. It is also to be appreciated that the various modes of treatment or prevention of medical conditions as described are intended to mean “substantial”, which includes total but also less than total treatment or prevention, and wherein some biologically or medically relevant result is achieved.

### I. Compositions

In one aspect, this disclosure provides modified stem cells that are capable of targeting glioma cells. In some embodiments, the modified stem cells comprise at least one controlled-release vehicle for the delivery of at least one anti-neoplastic agent to a glioma cell. The modified stem cells exhibit tropism to glioma cells. Thus, a locally-concentrated dose of the at least one anti-neoplastic agent is released, which may, for example, kill or damage the target cells or tissues, resulting in destruction of the tumor or reduction of the tumor size or volume and/or amelioration of one or more symptoms associated with the neoplasm. In this section, the components of the modified stem cells are described.

A. Stem Cells

Stem cells are an effective delivery vehicle for anti-neoplastic medicine to gliomas. While not wishing to be limited to theory, the directed migration abilities of stem cells provide an important component for using stem cells to deliver anti-neoplastic drugs. Many types of stem cells show a strong tropism toward gliomas, including, but not limited to, e.g., neural stem cells, bone marrow mesenchymal stem cells, and undifferentiated embryonic stem cells. See, e.g., Li et al, 2007, Neuroreport 18(17): 1821-1825; Aboody et al., 2000, Proc Nat Acad Sci USA. 97(23): 12846-12851.

In some embodiments, stem cells are obtained from a source tissue. Accordingly, whether a stem cell population is derived from adult or embryonic sources, the stem cells can be grown in a culture medium to increase the population of a heterogeneous mixture of cells, or a purified cell population. Several methods of growing stem cells outside of the body have been developed and are known in the art.

The stem cells to be expanded can be isolated from any organ of any mammalian organism, by any means known to one of skill in the art. The stem cells can be derived from embryonic or adult tissue. One of skill of the art can determine how to isolate the stem cells from the particular organ or tissue of interest, using methods known in the art. In one embodiment, the stem cells are isolated from umbilical cord blood. In one embodiment, the stem cells are isolated from bone marrow.

One of skill in the art will be able to determine a suitable growth medium for initial preparation of stem cells. Commonly used growth media for stem cells includes, but is not limited to, e.g., Iscove's modified Dulbecco's Media (IMDM) media, DMEM, KO-DMEM, DMEM/F12, RPMI 1640 medium, McCoy's 5A medium, minimum essential medium alpha medium (α-MEM), F-12K nutrient mixture medium (Kaighn's modification, F-12K), X-vivo 20, Stemline, CC100, H2000, Stemspan, MCDB 131 Medium, Basal Media Eagle (BME), Glasgow Minimum Essential Media, Modified Eagle Medium (MEM), Opti-MEM I Reduced Serum Media, Waymouth's MB 752/1 Media, Williams Media E, Medium NCTC-109, neuroplasma medium, BGJb Medium, Brinster's BMOC-3 Medium, CMRL Medium, CO₂-Independent Medium, Leibovitz's L-15 Media, and the like.

If desired, other components, such as growth factors, can be added. Exemplary growth factors and other components that can be added include, but are not limited to, thrombopoietin (TPO), stem cell factor (SCF), IL-1, IL-3, IL-7, flt-3 ligand (flt-3L), G-CSF, GM-CSF, Epo, FGF-1, FGF-2, FGF-4, FGF-20, IGF, EGF, NGF, LIF, PDGF, bone morphogenic proteins (BMP), activin-A, VEGF, forskolin, glucocorticoids, and the like. Furthermore, the media can contain either serum such as fetal calf, horse, or human serum, or serum substitution components. Numerous agents have been introduced into media to alleviate the need for serum. For example, serum substitutes have included bovine serum albumin (BSA), insulin, 2-mercaptoethanol and transferrin (TF).

The stem cells can then be stored for a desired period of time, if needed. Stem cell storage methods are known to those of skill in the art. The stem cells may be treated to a cryoprotection process, then stored frozen until needed. Cryoprotective agents are well known to one skilled in the art and can include, but are not limited to, dimethyl sulfoxide (DMSO), glycerol, polyvinylpyrrolidine, polyethylene glycol, albumin, dextran, sucrose, ethylene glycol, i-erythritol, D-ribitol, D-mannitol, D-sorbitol, i-inositol, D-lactose, or choline chloride as described in U.S. Pat. No. 6,461,645, which is incorporated by reference herein in its entirety.

The stem cells can be purified prior to contact with the controlled-release vehicle by methods known in the art, using, for example, antibody technology such as panning of cells, through the use of fluorescence activated cell sorting (FACS) methods, or magnet activated cell sorting methods such as that MACS apparatus, to isolate cells having the desired stem cell markers, or to remove unwanted, contaminating cell types having unwanted cell markers prior to contacting with controlled-release vehicle. Other methods of stem cell purification or concentration can include the use of techniques such as counterflow centrifugal elutriation, equilibrium density centrifugation, velocity sedimentation at unit gravity, immune rosetting and immune adherence, T lymphocyte depletion. Examples of stem cell markers that can be useful in purification include, but are not limited to, FLK-1, AC133, CD34, c-kit, CXCR-4, Oct-4, Rex-1, CD9, CD13, CD29, CD44, CD166, CD90, CD105, SH-3, SH-4, TRA-1-60, TRA-1-81, SSEA-4, Sox-2, and the like. Examples of cell surface markers that can be used as markers of contaminating, unwanted cell types depends on the stem cell phenotype sought. For example, if collection of pluripotent hematopoietic cells is desired, contaminating cells will possess markers of commitment to the differentiated hematopoietic cells such as CD38 or CD33. If selection of stromal mesenchymal cells is desired, then contaminating cells would be detected by expression of hematopoietic markers such as CD45. Additionally, stem cells can be purified based on properties such as size, density, adherence to certain substrates, or ability to efflux certain dyes such as Hoechst 33342 or Rhodamine 123.

In some embodiments, the stem cells are human mesenchymal stem cells (MSC). Mesenchymal stem cells are the formative pluripotent blast cells found in the bone marrow and peripheral blood. Mesenchymal stem cells are also commonly referred to as “marrow stromal cells” or just “stromal cells”. MSC can migrate toward glioma cells because of an inherent specific affinity for glioma cells (see Yuan et al., 2006. Cancer Res 66:2630-2638 and Nakamizo et al., 2005. Cancer Res 65:3307-3318).

Although MSCs are rare, comprising about 0.01-0.0001% of the total nucleated cells of bone marrow, the cells may be isolated from bone marrow, purified from other bone marrow cells, and expanded in culture without loss of their stem cell potential (Haynesworth S E et al. 1992. Bone 13, 81-88). In some embodiments, the MSC for use in the compositions and methods described herein can be isolated from peripheral blood or bone marrow. A method for preparing MSC has been described in U.S. Pat. No. 5,486,359. Furthermore, mesenchymal stem cells may also be isolated from umbilical cord blood, as described by Erices et al. 2000. Br. J Haematol 109(1):235-42. In some embodiments, the MSC are isolated from bone marrow or peripheral blood of the subject afflicted with a glioma who will be the recipient of the treatment, i.e., MSCs may be used in autologous transplantation.

Several techniques are known for the rapid isolation of mesenchymal stem cells including, but are not limited to, leucopheresis, density gradient fractionation, immunoselection, differential adhesion separation, and the like. For example, immunoselection can include isolation of a population of MSCs using monoclonal antibodies raised against surface antigens expressed by bone marrow-derived MSCs, i.e., SH2, SH3 or SH4, as described, for example, in U.S. Pat. No. 6,387,367. The SH2 antibody binds to endoglin (CD105), while SH3 and SH4 bind CD73. Further, these monoclonal antibodies provide effective probes which can be utilized for identifying, quantifying and purifying MSC, regardless of their source in the body. In one embodiment, MSCs are culture expanded to enrich for cells expressing CD45, CD73, CD105, stro-1, or a combination thereof. In another embodiment, human MSCs are culture-expanded to enrich for cells containing surface antigens identified by monoclonal antibodies SH2, SH3 or SH4, prior to administering the human MSCs to the subject. A stro-1 antibody is described in Gronthos et al., 1996, J. Hematother. 5: 15-23. Further cell surface markers that may be used to enrich for human MSCs, such as those found in Table I, page 237 of Fibbe et al., 2003. Ann. N.Y. Acad. Sci. 996: 235-244.

The MSC for use in the compositions and methods described herein can be maintained in culture media which can be chemically defined serum free media or can be a “complete medium”, such as Dulbecco's Modified Eagles Medium supplemented with 10% serum (DMEM). Examples of chemically defined serum free media are described in U.S. Pat. No. 5,908,782 and WO96/39487, and complete media are described in U.S. Pat. No. 5,486,359. Chemically defined medium comprises a minimum essential medium such as Iscove's Modified Dulbecco's Medium (IMDM), supplemented with human serum albumin, human Ex Cyte lipoprotein, transferrin, insulin, vitamins, essential and non-essential amino acids, sodium pyruvate, glutamine and a mitogen. These media stimulate MSC growth without differentiation. Culture for about 2 weeks results in 10 to 14 doublings of the population of adherent cells. After plating the cells, removal of non-adherent cells by changes of medium every 3 to 4 days results in a highly purified culture of adherent cells that have retained their stem cell characteristics, and can be identified and quantified by their expression of cell surface antigens identified by monoclonal antibodies SH2, SH3 and/or SH4.

In some embodiments, the stem cells are neural stem cells (NSC). NSCs can be isolated from post-natal and adult tissues. NSCs derived from post-natal and adult tissues are quantitatively equivalent with respect to their capacity to differentiate into neurons and glia, as well as in their growth and differentiation characteristics. However, the efficiency of in vitro isolation of NSCs from various post-natal and adult CNS can be much lower than isolation of NSCs from fetal tissues which harbor a more abundant population of NSCs.

The NSCs can be derived from one site and transplanted to another site within the same subject as an autograft. Furthermore, the NSCs can be derived from a genetically identical donor and transplanted as an isograft. Still further, the NSCs can be derived from a genetically non-identical member of the same species and transplanted as an allograft. Alternatively, NSCs can be derived from non-human origin and transplanted as a xenograft. With the use of immunosuppressants, allograft and xenograft of non-human neural precursors, such as neural precursors of porcine origin, can be grafted into human subjects.

A sample tissue can be dissociated by any standard method. In an embodiment, tissue is dissociated by gentle mechanical trituration using a pipet and a divalent cation-free saline buffer to form a suspension of dissociated cells. Sufficient dissociation to obtain largely single cells is desired to avoid excessive local cell density.

In an embodiment, a neural stem cell line can be induced to be further enriched for a particular subtype of neurons. A number of growth factors, chemicals, and natural substances have been screened to identify effective inducers of particular neurons such as tyrosine hydroxylase-expressing dopaminergic neurons and acetylcholine-producing cholinergic neurons from NSCs of midbrain or spinal cord. The factor or chemical or combination thereof can be introduced during the mitotic phase and/or the differentiation phase of the NSCs.

B. Controlled Release Vehicles

In some embodiments, the modified stem cells comprise at least one controlled release vehicle which includes at least one anti-neoplastic agent and at least one targeting moiety. In one embodiment, the controlled-release vehicle is carried by the stem cell to the glioma where the anti-neoplastic agent is released in an amount sufficient to destroy the tumor or reduce the tumor's size or volume, and/or ameliorate one or more symptoms/effects of the neoplasia. In one embodiment, the anti-neoplastic agent is released from the controlled-release vehicle in a greater amount or at a faster rate after the stem cell has migrated to the glioma.

Controlled-release vehicles useful in the present technology include, but are not limited to, e.g., nanoparticles; biocompatible polymers; polymeric matrices; liposomes; and lipospheres. The controlled-release vehicles provide a system for targeted and/or controlled-release drug delivery applications. More specifically, an anti-neoplastic agent may be enclosed within the controlled-release vehicle, which provides controlled release of the encapsulated anti-neoplastic agent. The controlled-release vehicle may incorporate targeting moieties displayed on their outer surfaces, so as to provide effective targeting of the controlled-release vehicle to the stem cell to assist in preparation of the modified stem cell compositions.

In some embodiments, the at least one controlled-release vehicle is a nanoparticle. A nanoparticle may be a physical structure such as a particle, nanoshell, nanocore, or nanosphere. A nanosphere is a particle having a solid spherical-type structure with a size of less than about 1,000 nanometers. A nanocore refers to a particle having a solid core with a size of less than about 1,000 nanometers. A nanoshell refers to a particle having a hollow core that is surrounded by a shell, such that the particle has a size of less than about 1,000 nanometers. When a nanoshell includes an anti-neoplastic agent, the anti-neoplastic agent is located in the core that is surrounded by the shell of the nanoshell. The nanoparticle may have any size, shape or morphology, for example, it may include spherical nanoparticles as well as non-spherical nanoparticles. In some embodiments, the nanoparticle has a size (diameter for spherical nanoparticles or length/width for non-spherical nanoparticles) of about 1 nm to about 1000 nm, or about 1 nm to about 500 nm, about 1 nm to about 300 nm, about 10 nm to about 300 nm, about 100 to about 300 nm, or about 150 nm to about 250 nm.

In one embodiment, the nanoparticles are silicate nanoparticles. Silicate nanoparticles include, but are not limited to, organically modified silicate nanoparticles loaded with an anti-neoplastic agent. Organically-modified silicate nanoparticles may include both hydrophobic and hydrophilic groups on the precursor alkoxy-organosilane to help the particles self-assemble both as normal micelles and reverse micelles under appropriate conditions. The resulting micellar (and reverse micellar) cores can be used for entrapping biomolecules like small molecule drugs, proteins, etc. This system be loaded with both hydrophilic as well as hydrophobic compounds, and it can be precipitated in oil-in-water microemulsions so that corrosive solvents like cyclohexane and complex purification steps like solvent evaporation, ultra-centrifugation, etc., can be avoided. The organic group of the nanoparticles can be further modified for the attachment of targeting moeities, and they may be biodegraded through the biochemical decomposition of the Si—C bond. The presence of the organic group also reduces the overall rigidity and density of the particle.

Nanoparticles of colloidal silica can be prepared by hydrolysis of tetraalkyl silane. This method, commonly referred to as the “sol-gel” method, can be further extended in the synthesis of organically modified silica particles, where the precursor alkoxysilane molecules also include one or two organic groups. The incorporation of organic groups modify the final structure of the silica network, e.g. leading to the formation of mesoporous matrices, characterized by a network structure of ordered and uniform porosity. Such porous matrices can host a number of biologically active molecules like fluorescent dyes, proteins, anti-neoplastic agents, image contrast agents, etc.

In one embodiment, at least one targeting moiety can be attached to the surface of the nanoparticles in order to target the nanoparticles to a stem cell. Thus, the surface of the nanoparticles can be functionalized with different antibodies or ligands in order to target the particles to stem cells containing surface antigens or ligand-specific receptors.

In one embodiment, the nanoparticle may be a layered silicate nanoparticle for the controlled release of the anti-neoplastic agent. Layered silicate nanoparticles can be selected from natural or synthetic layered silicate nanoparticles and may have a cross-sectional length (for instance, the diameter in the case of a spherical particle or the width in the case of a plate-shaped particle) between about 1 and about 1000 nanometers or from about 10 to about 500 nm or from about 150 to 350 nm. The spacing between the adjacent layers within the silicate nanoparticles may be in the range of about 5-20 angstroms.

Layered silicate nanoparticles can be selected from natural and synthetic versions of following: (a) allophane; (b) apophyllite; (c) bannisterite; (d) carletonite; (e) cavansite; (f) chrysocolla; (g) members of the clay group, including: (i) members of the chlorite group such as baileychlore, chamosite, the mineral chlorite, clinochlore, cookeite, nimite, pennantite, penninite, sudoite, (ii) glauconite, (iii) illite, (iv) kaolinite, (v) montmorillonite, (vi) palygorskite, (vii) pyrophyllite, (viii) sauconite, (ix) talc, and (x) vermiculite; (h) delhayelite; (i) elpidite; (j) fedorite; (k) franklinfurnaceite; (l) franklinphilite; (m) gonyerite; (n) gyrolite; (O) leucosphenite; (p) members of the mica group, including (i) biotite, (ii) lepidolite, (iii) muscovite, (iv) paragonite, (v) phlogopite, and (vi) zinnwaldite; (q) minehillite; (r) nordite; (s) pentagonite; (t) petalite; (u) prehnite; (v) rhodesite; (w) sanbornite; (x) members of the serpentine group, including (i) antigorite, (ii) clinochrysotile, (iii) lizardite, (iv) orthochrysotile and (v) serpentine; (y) wickenburgite; (z) zeophyllite; and mixtures thereof.

Additional layered silicate materials, not necessarily exclusive of those above, can be selected from natural and synthetic versions of the following: aliettite, swinefordite, yakhontovite, volkonskoite, stevensite, hectorite, magadiite, kenyaite, ledikite, laponite, saponite, sauconite, montmorillonite, bentonite, nontronite, beidellite, hectorite, other smectite group clays, and mixtures thereof.

Depending on the nature of the anti-neoplastic agent and the nature of the silicate nanoparticles, the therapeutic agent may be maintained in association with the layered silicate nanoparticles by any of a number of mechanisms including, for example, hydrogen bonding, Van de Waals bonding, bonding through hydrophilic/hydrophobic interactions, ionic bonding, and so forth. By associating the therapeutic agent with the silicate nanoparticles, each silicate particle becomes a depot for the therapeutic agent. In one embodiment, the therapeutic agent is associated with the silicate particle in a way such that it occupies the spaces between adjacent layers of the silicate particle.

In some embodiments, for example, where the therapeutic agent and the silicate nanoparticle are both of similar hydrophilicity (or are both of similar hydrophobicity), the therapeutic agent can spontaneously associate with layers of the silicate nanoparticles. In other embodiments, the silicate nanoparticles are surface-modified to carry various charges to bind certain molecules. For instance, in some embodiments, the silicate nanoparticles are modified to carry cationic charges or anionic charges. Moreover, in some embodiments, the silicate nanoparticles are modified to carry certain functional groups. For instance, a number of grafting techniques are known in the art for establishing various functional groups on the surfaces of silicate nanoparticles, including hydrophobic and ionic functional groups.

Once formed, the anti-neoplastic agent-loaded nanoparticles have great flexibility with respect to (a) the range of polymeric carriers into which they can be incorporated, and (b) the techniques by which they can be formulated into the polymeric carriers.

In some embodiments, polymeric carriers may be included with the anti-neoplastic agent to prolong release of the agent from the nanoparticle. For example, using gelatin in the shell assembly may prolong drug release. In one embodiment, the nanoshells are composed of multilayers of poly(dimethyldiallyl ammonium chloride) (PDDA) and gelatin. For biocompatibility considerations, PDDA may be replaced with cationic poly-L-lysine (PLL). Nanoshells fabricated from gelatin and PLL are both biocompatible and biodegradable. In some embodiments, therefore, the nanoshells are composed of biocompatible organic polymers, assembled by the electrostatic layer-by-layer (LbL) method. The shell diameter may be between 100 and 1500 nm or between 100 and 600 nm. The shell thickness may be between 10 and 100 nm or between 10 and 30 nm. Drug release kinetics may be varied by varying the nanoshell membrane properties (e.g., thickness, polymer identities, polymer molecular weights, and additives).

The polymers for use in the polymeric carriers may be homopolymers or copolymers (including alternating, random and block copolymers), they may be cyclic, linear or branched (e.g., polymers have star, comb or dendritic architecture), they may be natural or synthetic, they may be thermoplastic or thermosetting, and they may be hydrophobic, hydrophilic or amphiphilic.

Polymers for use in the polymeric carriers may be selected, e.g., from the following: polycarboxylic acid polymers and copolymers including polyacrylic acids; acetal polymers and copolymers; acrylate and methacrylate polymers and copolymers (e.g., n-butyl methacrylate); cellulosic polymers and copolymers, including cellulose acetates, cellulose nitrates, cellulose propionates, cellulose acetate butyrates, cellophanes, rayons, rayon triacetates, and cellulose ethers such as carboxymethyl celluloses and hydroxyalkyl celluloses; polyoxymethylene polymers and copolymers; polyimide polymers and copolymers such as polyether block imides, polyamidimides, polyesterimides, and polyetherimides; polysulfone polymers and copolymers including polyarylsulfones and polyethersulfones; polyamide polymers and copolymers including nylon 6,6, nylon 12, polycaprolactams and polyacrylamides; resins including alkyd resins, phenolic resins, urea resins, melamine resins, epoxy resins, allyl resins and epoxide resins; polycarbonates; polyacrylonitriles; polyvinylpyrrolidones (cross-linked and otherwise); polymers and copolymers of vinyl monomers including polyvinyl alcohols, polyvinyl halides such as polyvinyl chlorides, ethylene-vinylacetate copolymers (EVA), polyvinylidene chlorides, polyvinyl ethers such as polyvinyl methyl ethers, polystyrenes, styrene-maleic anhydride copolymers, styrene-butadiene copolymers, styrene-ethylene-butylene copolymers (e.g., a polystyrene-polyethylene/butylene-polystyrene (SEBS) copolymer, available as Kraton.RTM. G series polymers), styrene-isoprene copolymers (e.g., polystyrene-polyisoprene-polystyrene), acrylonitrile-styrene copolymers, acrylonitrile-butadiene-styrene copolymers, styrene-butadiene copolymers and styrene-isobutylene copolymers (e.g., polyisobutylene-polystyrene block copolymers such as SIBS), polyvinyl ketones, polyvinylcarbazoles, and polyvinyl esters such as polyvinyl acetates; polybenzimidazoles; polyalkyl oxide polymers and copolymers including polyethylene oxides (PEO); polyesters including polyethylene terephthalates and aliphatic polyesters such as polymers and copolymers of lactide (which includes lactic acid as well as d-,1- and meso lactide), epsilon-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, and 6,6-dimethyl-1,4-dioxan-2-one (a copolymer of polylactic acid and polycaprolactone is one specific example); polyether polymers and copolymers including polyarylethers such as polyphenylene ethers, polyether ketones, polyether ether ketones; polyphenylene sulfides; polyolefin polymers and copolymers, including polyalkylenes such as polypropylenes, polyethylenes (low and high density, low and high molecular weight), polybutylenes (such as polybut-1-ene and polyisobutylene), EPDM copolymers (e.g., santoprene), ethylene propylene diene monomer (EPDM) rubbers, poly-4-methyl-pen-1-enes, ethylene-alpha-olefin copolymers, ethylene-methyl methacrylate copolymers and ethylene-vinyl acetate copolymers; fluorinated polymers and copolymers, including polytetrafluoroethylenes (PTFE), poly(tetrafluoroethylene-co-hexafluoropropene) (FEP), modified ethylene-tetrafluoroethylene copolymers (ETFE), and polyvinylidene fluorides (PVDF); silicone polymers and copolymers; polyurethanes; p-xylylene polymers; polyiminocarbonates; copoly(ether-esters) such as polyethylene oxide-polylactic acid copolymers; polyphosphazines; polyalkylene oxalates; polyoxaamides and polyoxaesters (including those containing amines and/or amido groups); polyorthoesters; biopolymers, such as polypeptides, proteins, polysaccharides and fatty acids (and esters thereof), including fibrin, fibrinogen, collagen, elastin, chitosan, gelatin, starch, glycosaminoglycans such as hyaluronic acid; as well as blends and copolymers of the above.

Elastomeric polymers are useful in some embodiments. Among the elastomeric polymers are included (a) polyolefin polymers, for example, butyl containing polymers such as polyisobutylene, (b) polyolefin copolymers, for example, polyolefin-polyvinylaromatic copolymers such as polyisobutylene-polystyrene copolymers, poly(butadiene/butylene)-polystyrene copolymers, poly(ethylene/butylene)—polystyrene copolymers, and polybutadiene-polystyrene copolymers; and (c) silicone polymers and copolymers; as well as blends thereof. Specific examples of polyolefin-polyvinylaromatic copolymers include polyolefin-polyvinylaromatic diblock copolymers and polyvinylaromatic-polyolefin-polyvinylaromatic triblock copolymers, such as a polystyrene-poly(ethylene/butylene)-polystyrene (SEBS) triblock copolymer, available as Kraton®, and polystyrene-polyisobutylene-poly-styrene (SIBS) triblock copolymers, which are described, for example, in U.S. Pat. No. 5,741,331, U.S. Pat. No. 4,946,899 and U.S. Pat. No. 6,545,097, each of which is hereby incorporated by reference in its entirety. Additional polyolefin-polyvinylaromatic copolymers are set forth in the prior paragraph.

In some embodiments, the controlled-release vehicle is a liposome, liposphere, or lipid-based nanoparticle. The particles will encapsulate one or more anti-neoplastic agents, such as chemotoxins, proteins, peptides, antisense oligonucleotides, and carbohydrates that are used for the treatment or prevention of a medical condition characterized by a malignancy of the central nervous system. The anti-neoplastic agents are encapsulated within the aqueous core of the particles and are surrounded by a lipid bilayer or membrane that may comprise a primary phospholipid and a lysolipid. The primary phospholipid and the lysolipid may have the same or different acyl chain lengths.

A liposome is a spherical vesicle with an aqueous core and a membrane composed of a lipid bilayer that encapsulates one or more active agents within the aqueous core. A lipid bilayer is a membrane or zone of membrane composed of two opposing layers of lipid molecules. The molecules are arranged so that their hydrocarbon tails face one another to form an oily bilayer. The hydrocarbon tails are also referred to as “hydrocarbon chains” or “acyl chains.” The molecules have electrically charged or polar heads that face the aqueous core on one side of the membrane. The molecules of the lipid bilayer may comprise a primary phospholipid and a lysolipid. The primary phospholipid may have a hydrocarbon chain length that differs from the hydrocarbon chain length of the lysolipid. The primary phospholipid may have a chain length ranging from about 6 to 20 carbon atoms. The lysolipid may have a chain length ranging from about 6 to 24 carbon atoms. In one embodiment, the difference in chain length between the primary phospholipid and lysolipid is about 4 carbon atoms.

The primary phospholipid may comprise a di-chain phospholipid, for example, 1,2-dipalmitoyl-sn-glycero-3-phosphocholine (DPPC) or 1,2-di-distearoyl-sn-glycero-3-phosphocholine (DSPC). The primary phospholipid may also comprise a tri-chain phospholipid, or any other phospholipid.

The lysolipid may comprise a molecule with a single acyl chain. The molecule may be a derivative of a phosphatic acid that lacks one of its fatty acid chains due to hydrolytic removal. The lysolipid may comprise a C6-C20 monoacyl lysolipid, and a surface active agent such as 1-myristoyl-2-hydroxy-sn-glycero-3-phosphocholine (MMPC) or 1-palmitoyl-2-hydroxy-s/n-glycero-3-phosphocholine (MPPC). Other examples of lysolipids include 1-oleoyl-2-hydroxy-sn-glycero-3-phosphocholine (MOPC), 1-lauroyl-2-hydroxy-sn-glycero-3-phosphocholine (MLPC), and 1-stearoyl-2-hydroxy-sn-glycero-3-phosphocholine (MSPC), or any other mono-chain lysolipid.

The primary phospholipid and the lysolipid organize to form a lipid bilayer. The primary phospholipid may comprise from about 80% to 95% of the bilayer. The molar ratio of the primary phospholipid to the lysolipid may range from about 80:20 to about 95:5. The bilayer may also contain other substances such as cholesterol, a surface coating of polyethylene glycol, or another polymer such as dextran.

The rate of release of the anti-neoplastic agent from the compositions can be controlled by adjusting the thickness, particle size, structure, type of polymer used, and density of the layers of polymers in the controlled release vehicles. Density of the polymer coating can be adjusted by varying the loading of the anti-neoplastic agent in the coating. When the coating contains no anti-neoplastic agent, the polymer coating is densest, and the elution of the anti-neoplastic agent through the coating is slowest. By contrast, when anti-neoplastic agent is loaded into the coating, the coating becomes porous once the anti-neoplastic agent has eluted out, starting from the outer surface of the coating and, therefore, the active agent(s) at the center of the particle can elute at an increased rate. The higher the drug loading in the coating layer, the lower the density and the higher the elution rate. The loading of anti-neoplastic agent in the coating can be lower than that in the interior of the particles beneath the exterior coating. Release rate of anti-neoplastic from the particles can also be controlled by mixing particles with different release rates prepared as described above. In one embodiment, the anti-neoplastic agent has a release rate of about 1 to 5 days, about 1 to 7 days, about 1 to 14 days, about 1 to 31 days, about 5 to 14 days, about 5 to 31 days, or about 5 to 60 days. In one embodiment, the anti-neoplastic agent begins to release from the controlled-release vehicle after the stem cell has migrated to the site of the glioma.

C. Anti-Neoplastic Agents

In some embodiments, the controlled-release vehicle comprises at least one anti-neoplastic agent useful in the treatment or prevention of a medical condition associated with a malignancy or neoplasm of the central nervous system, e.g., a glioma. In one embodiment, the anti-neoplastic agent is a small molecule drug or other biological effector molecule. For example, the therapeutic agent may be a biological effector molecule which has activity in a biological system. Biological effector molecules, include, but are not limited to, a protein, polypeptide, or peptide, including, but not limited to, a structural protein, an enzyme, a cytokine (such as an interferon and/or an interleukin), a polyclonal or monoclonal antibody, or an effective part thereof, such as an Fv fragment, which antibody or part thereof, may be natural, synthetic or humanized, a peptide hormone, a receptor, or a signaling molecule. Included within the term “immunoglobulin” are intact immunoglobulins as well as antibody fragments such as Fv, a single chain Fv (scFv), a Fab or a F(ab′)₂.

Anti-neoplastic agents of interest include, without limitation, pharmacologically active drugs, genetically active molecules, etc. Compounds of interest include chemotherapeutic agents, anti-inflammatory agents, hormones or hormone antagonists, ion channel modifiers, and neuroactive agents. Examples of pharmaceutical agents include those described in, “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming organs; Hormones and Hormone Antagonists; Vitamins, Dermatology; and Toxicology, all incorporated herein by reference. Also included are toxins, and biological and chemical warfare agents, for example see Somani, S. M. (ed.), Chemical Warfare Agents, Academic Press, New York (1992)).

In some embodiments, the biological effector molecules are immunoglobulins, antibodies, Fv fragments, etc., which are capable of binding to antigens in an intracellular environment. These types of molecules are known as “intrabodies” or “intracellular antibodies.” An “intracellular antibody” or an “intrabody” is an antibody which is capable of binding to its target or cognate antigen within the environment of a cell, or in an environment which mimics an environment within the cell. Selection methods for directly identifying such “intrabodies” include the use of an in vivo two-hybrid system for selecting antibodies with the ability to bind to antigens inside mammalian cells. Such methods are described in PCT/GB00/00876, incorporated herein by reference. Techniques for producing intracellular antibodies, such as anti-β-galactosidase scFvs, have also been described in Martineau et al., J Mol Biol 280:117-127 (1998) and Visintin et al., Proc. Natl. Acad. Sci. USA 96:11723-1728 (1999).

In some embodiments, the biological effector molecule is selected from the group consisting of a protein, a polypeptide, a peptide, a nucleic acid, a virus, a virus-like an amino acid, an amino acid analogue, a modified amino acid, a modified amino acid analogue, a steroid, a proteoglycan, a lipid and a carbohydrate or a combination thereof (e.g., chromosomal material comprising both protein and DNA components or a pair or set of effectors, wherein one or more convert another to active form, for example catalytically).

A biological effector molecule may include a nucleic acid, including, but not limited to, an oligonucleotide or modified oligonucleotide, an antisense oligonucleotide or modified antisense oligonucleotide, an aptamer, a cDNA, genomic DNA, an artificial or natural chromosome (e.g., a yeast artificial chromosome) or a part thereof, RNA, including an siRNA, a shRNA, mRNA, tRNA, rRNA or a ribozyme, or a peptide nucleic acid (PNA); a virus or virus-like particles; a nucleotide or ribonucleotide or synthetic analogue thereof, which may be modified or unmodified. The biological effector molecule can also be an amino acid or analogue thereof, which may be modified or unmodified or a non-peptide (e.g., steroid) hormone; a proteoglycan; a lipid; or a carbohydrate.

Small molecules, including inorganic and organic chemicals, may also be used. In one embodiment, the small molecule is a pharmaceutically active agent. Useful classes of pharmaceutically active agents include, but are not limited to, antibiotics, anti-inflammatory drugs, angiogenic or vasoactive agents, growth factors and chemotherapeutic agents (e.g., tumour suppressors).

As used herein, the term “prodrug” refers to any substance that is converted in vivo into a different substance which has the desired pharmaceutical activity. If a prodrug is loaded into the controlled-release vehicle in an inactive form, a second effector molecule may also be loaded. Such a second effector molecule is usefully an activating polypeptide which converts the inactive prodrug to active drug form. In one embodiment, activating polypeptides include, but are not limited to, viral thymidine kinase (encoded by Genbank Accession No. J02224), carboxypeptidase A (encoded by Genbank Accession No. M27717), α-galactosidase (encoded by Genbank Accession No. M13571), β-gluucuronidase (encoded by Genbank Accession No. M15182), alkaline phosphatase (encoded by Genbank Accession No. J03252 J03512), or cytochrome P-450 (encoded by Genbank Accession No. D00003 N00003), plasmin, carboxypeptidase G2, cytosine deaminase, glucose oxidase, xanthine oxidase, β-glucosidase, azoreductase, t-gutamyl transferase, β-lactamase, or penicillin amidase.

The therapeutic agent may also be a radiotherapeutic agent, such as for example a compound or complex of boron or gadolinium, useful in neutron capture therapy, or an inherently radioactive isotope such as ⁵⁵Fe or ¹²⁵I or ¹³¹I.

Anti-neoplastic agents useful in the compositions and methods of the present technology, include, but are not limited to, e.g., asparaginase; adriamycin; alkaloids; alkylating agent; altretamine; amsacrine; anti-metabolite compound; antitumour antibiotics; azathioprine; bleomycin sulfate; busulfan; camptothecins; carboplatin; carmustine; chlorambucil; cisplatin; cladribine; cyclophosphamide; Cytarabine; Dacarbazine; dactinomycin; daunorubicin; docetaxel; doxorubicin hydrochloride; epipodophyllotoxins; epirubicin hydrochloride; estramustine sodium phosphate; etoposide; etoposide phosphate; finasteride; fludarabine phosphate; fluorouracil; gonadotropin-releasing hormone agonists (GnRH); Goserelin; hydroxyurea; idarubicin hydrochloride; ifosfamide; irinotecan; lomustine; marimastat; mechlorethamine; mechlorethamine hydrochloride; melphalan; mercaptopurine; methotrexate sodium; mitomycin; mitotane; mitoxantrone hydrochloride; oxaliplatin; paclitaxel; Podophyllotoxin; porfimer sodium; procarbazine hydrochloride; radiotherapeutics; streptozocin; suramin; tamoxifen; taxanes; taxol; teniposide terpenoids; thalidomide; Thioguanine; thiotepa; TNP 470; topoisomerase inhibitors; topotecan; tretinoin (all-trans retinoic acid); vinblastine; vinblastine sulfate; vinca alkaloid; vincristine; vincristine sulfate; Vindesine; vindesine sulfate; and vinorelbine tartrate.

The anti-neoplastic agent(s) within the release regions need not be restricted to the location of the drug loaded nanoparticles. For example, in addition to being present within the drug loaded nanoparticles, anti-neoplastic agent(s) can also be dissolved or dispersed within the carrier that surrounds the modified stem cells, as desired.

D. Targeting Moieties

In some embodiments, the controlled release vehicles comprise one or more targeting moieties for the selective binding of the controlled release vehicle to a target molecule. In one embodiment, the targeting moiety specifically binds to target molecules of a stem cell. Examples of targeting moieties include, but are not limited to, e.g., an antigen; ligand; receptor; one member of a specific binding pair; polyamide; peptide; carbohydrate; oligosaccharide; polysaccharide; low density lipoprotein (LDL) or an apoprotein of LDL; steroid; steroid derivative; hormone; hormone-mimic; lectin; drug; antibiotic; aptamer; DNA; RNA; lipid; an antibody; and an antibody-related polypeptide. In some embodiments, the targeting moiety is an antibody or antibody-related polypeptide. For example, antibodies useful as targeting moieties include antibodies in general and monoclonal antibodies. The targeting moiety can include a polypeptide having an affinity for a polysaccharide target, for example, a lectin (such as a seed, bean, root, bark, seaweed, fungal, bacterial, or invertebrate lectin).

Many targeting moieties and methods for targeting compounds are well known to those of skill in the art. For non-limiting examples of targeting methods, See, e.g., U.S. Pat. Nos. 6,316,652; 6,274,552; 6,271,359; 6,253,872; 6,139,865; 6,131,570; 6,120,751; 6,071,495; 6,060,082; 6,048,736; 6,039,975; 6,004,534; 5,985,307; 5,972,366; 5,900,252; 5,840,674; 5,759,542 and 5,709,874.

The targeting moiety may be an antibody or an antigen binding antibody fragment capable of specifically binding to at least one epitope on the target molecule(s) associated with, produced by or on the surface of a stem cell. The antibody or antibody fragment may be monospecific or multispecific. Both polyclonal and monoclonal antibodies may be used, as well as certain recombinant antibodies, such as chimeric and humanized antibodies and fusion proteins.

The targeting moiety may be multivalent and/or multispecific. By “multivalent” it is meant that the targeting moiety may bind more than one target, which may have the same or a different structure, simultaneously. By “multispecific” it is meant that the subject agents may bind simultaneously to at least two targets which are of different structure. For example, a targeting moiety having two different specificities would be considered multivalent and multispecific because it can bind two structurally different targets simultaneously. On the other hand, a molecule which having two or more specific arms which bind the same target, but no other specificities, would be multivalent but not multispecific.

In some embodiments, the targeting moiety is a stem cell specific surface antibody. In one embodiment, the antibody or fragment thereof specifically binds an epitope of CD90, CD105, or CD73, which are specific to mesenchymal stem cells. In one embodiment, the antibody or fragment thereof specifically binds an epitope of CD3, CD5, CD7, CD10, CD 11b, CD13, CD14, CD16, CD19, CD20, CD22, CD23, CD25, CD31, CD33, CD41, CD45, CD54, CD80, CD83, CD86, TAPA-1, CD15, CD95, CD9, CD8, CD34, CD38, CD56, CD81, CD152, CD133, CD117, CD154, which are present on neural stem cells. In one embodiment, the antibody or fragment thereof specifically binds an epitope of CD9, CD29, CD34, CD44, CD45, CD49e, CD54, CD71, CD90, CD105, CD106, CD120a, CD124, CD166, Sca-1, SH2, SH3, which are present on embryonic stem cells.

E. Markers for Modified Stem Cells

The modified stem cells may also be labeled with one or more positive markers that can be used to monitor over time the number or concentration of modified stem cells in subject or in culture. It is anticipated that the overall number of modified stem cells will decay over time following initial administration. As such, it may be appropriate to monitor the signal from one or more positive markers. There are presently several fluorescent compounds, for example, that are approved by the Food & Drug Administration for human use including but not limited to fluorescein, indocyanin green, and rhodamine B. For example, stem cells may be specifically labeled with fluorescein isothiocyanate (FITC; Bratosin et al., Cytometry 46:351-356 (2001)).

Other dyes may be useful for tracking modified stem cells in human and non-human circulation. A number of reagents may be used to label a stem cell. For example, VivoTag 680 (VT680; V isEn Medical, Woburn, Mass., USA) may be used to track cells in vivo. VT680 is a near-infrared fluorochrome with a peak excitation wavelength of 670±5 nm and a peak emission wavelength of 688±5 nm. VT680 also contains an amine reactive NHS ester which enables it to cross-link with proteins and peptides. As such, the surface of cells may be labeled with VT680 (See, e.g., Swirski, et al., (2007) PloS ONE 10:e1075). For example, 4×10⁶ cells/ml are incubated with VT680 diluted in complete culture medium at a final concentration of 0.3 to 300 μg/ml for 30 min at 37° C. The cells are washed twice with complete culture medium after labeling. Cells may be non-specifically labeled based on proteins expressed on the surface of the modified stem cell. Alternatively, a specific protein may be labeled with VT680. In some instances, an antibody directed against a specific protein associated with the stem cell may be used to selectively label cells. In other instances, a protein or peptide may be directly labeled with VT680 ex vivo and subsequently either attached to the surface of the cell or incorporated into the interior of the cell.

Alternatively, a stem cell may be labeled with other red and/or near-infrared dyes including, for example, cyanine dyes such as Cy5, Cy5.5, and Cy7 (Amersham Biosciences, Piscataway, N.J., USA) and/or a variety of Alexa Fluor dyes including Alexa Fluor 633, Alexa Fluor 635, Alexa Fluor 647, Alexa Fluor 660, Alexa Fluor 680, Alexa Fluor 700 and Alexa Fluor 750 (Molecular Probes-Invitrogen, Carlsbad, Calif., USA). Additional fluorophores include IRD41 and IRD700 (LI-COR, Lincoln, Nebr., USA), NIR-1 and 1C5-OSu (Dejindo, Kumamotot, Japan), LaJolla Blue (Diatron, Miami, Fla., USA), FAR-Blue, FAR-Green One, and FAR-Green Two (Innosense, Giacosa, Italy), ADS 790-NS and ADS 821-NS (American Dye Source, Montreal, CA). Quantum dots (Qdots) of various emission/excitation properties may also be used for labeling cells (See, e.g., Jaiswal et al., Nature Biotech. 21:47-51 (2003)). Many of these fluorophores are available from commercial sources either attached to primary or secondary antibodies or as amine-reactive succinimidyl or monosuccinimidyl esters, for example, ready for conjugation to a protein or proteins either on the surface or inside the stem cells.

Magnetic nanoparticles may be used to track cells in vivo using high resolution MRI (Montet-Abou et al., Molecular Imaging 4:165-171 (2005)). Magnetic particles may be internalized by several mechanisms. Magnetic particles may be taken up by a cell through fluid-phase pinocytosis or phagocytosis. Alternatively, the magnetic particles may be modified to contain a surface agent such as, e.g., the membrane translocating HIV tat peptide which promotes internalization. In some instances, a magnetic nanoparticle such as, for example, Feridex IV®, an FDA approved magnetic resonance contrast reagent, may be internalized into hematopoietic cells in conjunction with a transfection agent such as, for example, protamine sulfate (PRO), polylysine (PLL), and lipofectamine (LFA).

F. Preparation of the Modified Stem Cells

In one embodiment, assembly of the modified stem cells begins with a suspension of the controlled-release vehicle, such as a colloidal suspension of polymeric nanoparticles. The nanoparticle cores may be constructed of any solid material that has (or can be given) a surface charge, and which can be dissolved after the shell layers have been formed without disrupting the layered shell coating. Core materials include, but are not limited to, melamine formaldehyde, poly(lactic acid-co-lysine), amino- and carboxy-substituted polycarbonates, polyesters, polyacetals, polyacrylates, and polystyrenes, and various copolymers thereof, as well as inorganic core materials such as colloidal silica, titania, or zirconia, or finely divided metallic oxides and carbonates such as MnCO₃ microcrystals. For example, commercially available monodisperse polystyrene, poly(methyl methacrylate), or melamine formaldehyde particles may be used as cores for hollow nanoshell formation. If necessary, the particles are reduced in size by an appropriate means, for example by partial dissolution, decomposition, or erosion, before they are used as cores for nanoshell fabrication. Surface charges, if not already present in the cores, may be introduced by methods known in the art, for example by coating with a layer of charged polymer, or by surface oxidation and/or coupling of charged chemical moieties. See for example Surface-Controlled Nanoscale Materials for High-Added-Value Applications, K. E. Gonsalves et al., Eds, 1998, Materials Research Society (Warrendale, Pa.), and Synthesis, Functionalization and Surface Treatment of Nanoparticles, M.-I. Baraton, Ed., 2003, American Scientific Publishers (Stevenson Ranch, Calif.).

In some embodiments, the outermost shell further comprises targeting moieties such as proteins, peptides, ligands, and antibodies. Targeting moieties include, but are not limited to, e.g., peptides such as homing peptides, proteins, receptor-specific ligands and stem cell-specific antibodies (e.g., CD90). Incorporation of a targeting moiety into the outer shell may be accomplished by any of the methods known in the art of targeted drug delivery. Methods include, but are not limited to, e.g., covalent attachment of a targeting moiety to one or more components of the outermost shell, either directly or via linkers, binding of biotinylated targeting moieties to avidin or streptavidin molecules attached to the outer shell, and electrostatic binding of appropriately charged molecules, such as the antibodies in the examples below. These and other methods are well known in the art; see, e.g., A. Coombes et al., 1997. Biomaterials 18:1153-1161.

For covalent attachment, chemically reactive groups present on the targeting moiety and on the outer layer of the controlled release vehicle (e.g., nanoparticle) may be coupled to one another by means known in the art. The amino groups provided by the lysine groups of gelatin, for example, can be coupled with activated targeting moieties, such as those where carbodiimides have been used as activating agents for carboxyl groups, rendering them reactive with amino groups. In an alternative embodiment, avidin or streptavidin may be covalently bound to the outer surface of the nanoshells, and biotinylated targeting moieties can then be coupled to the nanoshell surface efficiently. (Wilchek, et al., Meth. Enzmol., 184:5-13, (1990)). Similarly, protein A can be incorporated into the outer shell of the nanospheres and used to bind immunoglobulin targeting moieties.

The controlled-release vehicle and targeting moiety can be conjugated, directly or through a linking component. For example, molecules that contain carboxyl groups can be joined to lysine-amino groups in the target polypeptides either by preformed reactive esters (such as N-hydroxy succinimide ester) or esters conjugated in situ by a carbodiimide-mediated reaction. The same applies to molecules that contain sulfonic acid groups, which can be transformed to sulfonyl chlorides which react with amino groups. Molecules that have carboxyl groups can be joined to amino groups, such as on a polypeptide, by an in situ carbodiimide method. Molecules can also be attached to hydroxyl groups of serine or threonine residues or to sulfhydryl groups of cysteine residues.

Methods of joining components can use heterobifunctional cross linking reagents. These agents bind a functional group in one chain and to a different functional group in the second chain. These functional groups include amino, carboxyl, sulfhydryl, and aldehyde. There are many permutations of appropriate moieties which will react with these groups and with differently formulated structures, to conjugate them together. (See Merrifield et al., Ciba Found Symp. 186: 5-20 (1994)).

Reactive groups and classes of reactions useful in preparing the disclosed conjugates are generally those that are well known in the art of bioconjugate chemistry. Classes of reactions include those that proceed under relatively mild conditions. These include, but are not limited to, e.g., nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction). These and other useful reactions are discussed in, for example, Morrison et al., Organic Chemistry, 4th Ed., Allyn and Bacon, Inc. (1983), and Hermanson, Bioconjugate Techniques, Academic Press, San Diego (1996).

For example, useful reactive functional groups include: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups, which can be converted to esters, ethers, aldehydes, etc.; (c) haloalkyl groups, wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups, which are capable of participating in Diels-Alder reactions such as, for example, maleimido groups; (e) carbonyl groups, such that subsequent derivatization is possible via formation of carbonyl derivatives such as, for example, imines, hydrazones, semicarbazones or oximes, or via such mechanisms as Grignard addition or alkyllithium addition; (f) sulfonyl groups for subsequent reaction with amines, for example, to form sulfonamides; (g) thiol groups, which can be converted to disulfides or reacted with acyl halides; (h) amine or sulfhydryl groups, which can be, for example, acylated, alkylated or oxidized; (i) alkenes, which can undergo, for example, cycloadditions, acylation, Michael addition, etc; (j) epoxides, which can react with, for example, amines and hydroxyl compounds; and (k) phosphoramidites and other standard functional groups useful in nucleic acid synthesis.

In one embodiment, the controlled release vehicle is contacted with a stem cell, whereupon it enters the stem cell by endocytosis or pinocytosis or remains attached to the surface of the stem cell through specific binding of the targeting moiety and the corresponding binding partner. The modified stem cells may then be introduced into a subject.

In another embodiment, the controlled release vehicle is attached to the stem cell via a covalent attachment. For example, the targeting moiety may be derivatized and bound to the stem cell using a coupling compound containing an electrophilic group that will react with nucleophiles on the stem cell to form the interbonded relationship. Representative of these electrophilic groups are α, β unsaturated carbonyls, alkyl halides and thiol reagents such as substituted maleimides. In addition, the coupling compound can be coupled to the targeting moiety via one or more of the functional groups in the targeting moiety such as amino, carboxyl and tryosine groups. For this purpose, coupling compounds should contain free carboxyl groups, free amino groups, aromatic amino groups, and other groups capable of reaction with enzyme functional groups. Highly charged derivatives of targeting moiety can also be prepared for immobilization on stem cells through electrostatic bonding. Examples of these derivatives would include polylysyl and polyglutamyl enzymes.

The choice of the reactive group embodied in the derivative depends on the reactive conditions employed to couple the electrophile with the nucleophilic groups on the stem cell for immobilization. A factor is the desire not to inactivate the coupling agent prior to coupling of the targeting moiety immobilized by the attachment to the stem cell.

Such coupling immobilization reactions can proceed in a number of ways. A coupling agent can be used to form a bridge between the macromolecule and the stem cell. In this case, the coupling agent should possess a functional group such as a carboxyl group which can be caused to react with the targeting moiety. One pathway for preparing the macromolecular derivative comprises the utilization of carboxyl groups in the coupling agent to form mixed anhydrides which react with the targeting moiety, in which use is made of an activator which is capable of forming the mixed anhydride. Representative of such activators are isobutylchloroformate or other chloroformates which give a mixed anhydride with coupling agents such as 5,5′-(dithiobis (2-nitrobenzoic acid) (DTNB), p-chloromercuribenzoate (CMB), or m-maleimidobenzoic acid (MBA). The mixed anhydride of the coupling agent reacts with the targeting moiety to yield the reactive derivative which in turn can react with nucleophilic groups on the stem cell to immobilize the macromolecule.

Functional groups on the targeting moiety such as carboxyl groups can be activated with carbodiimides and the like activators. Subsequently, functional groups on the bridging reagent, such as amino groups, will react with the activated group on the targeting moiety to form the reactive derivative. In addition, the coupling agent should possess a second reactive grouping which will react with appropriate nucleophilic groups on the stem cell to form the bridge. Typical of such reactive groupings are alkylating agents such as iodoacetic acid, α, β unsaturated carbonyl compounds, such as acrylic acid and the like, thiol reagents, such as mercurials, substituted maleimides and the like.

Alternatively, functional groups on the targeting moiety can be activated so as to react directly with nucleophiles on stem cells to obviate the need for a bridge-forming compound. For this purpose, use is made of an activator such as Woodward's Reagent K or the like reagent which brings about the formation of carboxyl groups in the targeting moiety into enol esters, as distinguished from mixed anhydrides. The enol ester derivatives of targeting moieties will subsequently react with nucleophilic groups on the stem cell to effect immobilization of the macromolecule.

G. Formulations of Pharmaceutical Compositions

The modified stem cells can be incorporated into pharmaceutical compositions suitable for administration. The pharmaceutical compositions generally comprise substantially purified modified stem cells and a pharmaceutically-acceptable carrier in a form suitable for administration to a subject. Pharmaceutically-acceptable carriers are determined in part by the particular composition being administered, as well as by the particular method used to administer the composition. Accordingly, there is a wide variety of suitable formulations of pharmaceutical compositions for administering the modified stem cells (See, e.g., Remington's Pharmaceutical Sciences, Mack Publishing Co., Easton, Pa. 18^{th} ed. (1990)). The pharmaceutical compositions are generally formulated as sterile, substantially isotonic and in full compliance with all Good Manufacturing Practice (GMP) regulations of the U.S. Food and Drug Administration.

The terms “pharmaceutically-acceptable,” “physiologically-tolerable,” and grammatical variations thereof, as they refer to compositions, carriers, diluents and reagents, are used interchangeably and represent that the materials are capable of administration to or upon a subject without the production of undesirable physiological effects to a degree that would prohibit administration of the composition. For example, “pharmaceutically-acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous. A person of ordinary skill in the art, is able to determine the appropriate timing, sequence and dosages of administration for particular drugs and compositions.

Examples of such carriers or diluents include, but are not limited to, water, saline, Ringer's solutions, dextrose solution, and 5% human serum albumin. The use of such media and compounds for pharmaceutically active substances is well known in the art. Except insofar as any conventional media or compound is incompatible with the modified stem cells, use thereof in the compositions is contemplated. Supplementary active compounds can also be incorporated into the compositions.

A pharmaceutical composition is formulated to be compatible with its intended route of administration. The modified stem cells can be administered by parenteral, topical, intravenous, oral, subcutaneous, intraarterial, intradermal, transdermal, rectal, intracranial, intrathecal, intraperitoneal, intranasal, intramuscular route or as inhalants. The modified stem cells can optionally be administered in combination with other agents that are at least partly effective in treating various diseases.

In an embodiment, upon harvesting of the cells, the modified cells are concentrated by brief centrifugation. The cells can be further washed and re-suspended in a final, clinically usable solution such as saline, buffered saline, or, alternatively, be re-suspended in a storage or hibernation solution. Alternatively, the cells can be re-suspended in a freezing medium such as media plus dimethylsulfoxide, or any other suitable cryoprotectant, and frozen for storage.

The solution is formulated to maintain the viability of live cells for a prolonged period of time. In an embodiment, the storage solution can be adapted to be used for shipping live cells in a ready-to-use formulation to a transplantation surgery site for immediate use. Suitable conditions for shipping live cells to a distant site also includes an insulation device that can maintain a stable temperature range between about 0° C. and about 20° C. for at least 24 hours. Live cells stored at between about 0° C. and about 8° C. for about 24 hours to about 48 hours are engraftable for treatment of a disease or condition.

In an embodiment, the cells are concentrated in a solution such as the clinically usable solutions described above. In an embodiment, the cells are concentrated to an appropriate cell density which can be the same or different from the cell density for administration of the cells. In an embodiment, the cell density for administration can vary from about 1,000 cells per microliter to about 1,000,000 cells per microliter depending upon factors such as the site of the injection, the neurodegenerative status of the injection site, the minimum dose necessary for a beneficial effect, and toxicity side-effect considerations. In an embodiment, the disclosed methods include injecting cells at a cell density of about 5,000 to about 50,000 cells per microliter.

The volume of media in which the expanded cells are suspended for delivery to a treatment area can be referred to herein as the injection volume. The injection volume depends upon the injection site and the state of the tissue. More specifically, the lower limit of the injection volume can be determined by practical liquid handling of viscous suspensions of high cell density as well as the tendency of the cells to cluster. The upper limit of the injection volume can be determined by limits of compression force exerted by the injection volume that are necessary to avoid injuring the host tissue, as well as the practical surgery time.

Sterile injectable solutions can be prepared by incorporating the modified stem cells in the required amount in an appropriate medium with one or a combination of ingredients enumerated above, as required. Generally, dispersions are prepared by incorporating the modified stem cells into a sterile vehicle that contains a basic dispersion medium and the required other ingredients from those enumerated above. The modified stem cells can be administered in the form of a depot injection or implant preparation which can be formulated in such a manner as to permit a sustained or pulsatile release of the active ingredient.

In one embodiment, the modified stem cells are prepared with carriers that will protect the modified stem cells against rapid elimination from the body, such as a controlled release formulation, including implants and microencapsulated delivery systems.

### II. Methods

This section will generally describe some embodiments of the methods of using the modified stem cell compositions. Further details of particular applications are described in the sections that follow.

In one aspect, the disclosure provides methods of using a modified stem cell to carry and release an anti-neoplastic agent in proximity to a target cell (e.g., a glioma cell). Thus, the tropism of stem cells for glioma cells is used to deliver anti-neoplastic agents to the tumor. In one embodiment, the modified stem cells may be administered to a subject after surgery. In one embodiment, the modified stem cells may be administered to a subject who, for one or more reasons, has not and/or cannot undergo surgery or conventional radiotherapy or chemotherapy.

The modified stem cells may be used to target glioma cells and deliver an anti-neoplastic agent to damage or destroy the glioma cells. For instance, the modified stem cells may comprise stem cells loaded with one or more therapeutic agents, e.g., an anti-neoplastic agent, which is released from the modified stem cell at the desired location. Consequently, the neoplastic cell is brought into close contact with a relatively high concentration of the agent. As described above, the modified stem cells may be loaded with one or more chemotherapeutic agents for targeted delivery to a neoplastic cell.

The disclosure provides for methods of administering or transplanting the modified stem cell to a subject. A modified stem cell is administered to a subject when it is transferred from a vessel into a patient. Administering can include the steps of isolating a stem cell and transplanting the stem cell into a subject. Transplantation can involve transferring a stem cell into a subject by injection of a cell suspension into the subject, surgical implantation of a cell mass into a tissue or organ of the subject, or perfusion of a tissue or organ with a cell suspension. The route of transferring the stem cell or transplantation, will be determined by the need for the cell to reside in a particular tissue or organ and by the ability of the cell to find and be retained by the desired target tissue or organ. In the case where a transplanted cell is to reside in a particular location, it can be surgically placed into a tissue or organ or simply injected into the bloodstream if the cell has the capability to migrate to the desired target organ.

By way of example, a patient in need of modified stem cells as described herein can be treated as follows. Modified stem cells can be administered to the patient, e.g., in a biologically compatible solution or a pharmaceutically acceptable delivery vehicle, by injection or any number of other methods. One embodiment is injection into the cranium of the subject. The dosages administered will vary from patient to patient; a “therapeutically effective dose” can be determined, for example but not limited to, by the level of reduction in tumor size or volume. Generally, a composition including stem cells will be administered in a single dose in the range of 10⁵-10⁸ cells per kg body weight, or in the range of 10⁶-10⁷ cells per kg body weight. This dosage may be repeated daily, weekly, monthly, yearly, or as considered appropriate by the treating physician. Cell populations can also be removed from the patient or otherwise provided, expanded ex vivo, contacted with a controlled-release vehicle containing an anti-neoplastic agent, and then reintroduced into the patient.

In one aspect, the disclosure provides one or more methods of modifying stem cells including contacting a stem cell with one or more controlled release vehicles. In some embodiments, the targeting moieties of the controlled-release vehicles are designed to recognize one or more stem cells.

### EXAMPLES

The present technology is further illustrated by the following examples, which should not be construed as limiting in any way.

### Example 1

### Preparation of Silicate Nanoparticles Loaded with Anti-Neoplastic Agent

Preparation of Hollow Mesoporous Silica Spheres as a Drug Delivery System. Hollow mesoporous silica (HMS) spheres were prepared by the method as described in Li et al., “Hollow spheres of mesoporous aluminosilicate with a three-dimensional pore network and extraordinarily high hydrothermal stability.” Nano Lett. 3:609-612 (2003). The drug-storage profile was taken as described in Vallet-Regi et al., Chem. Mater 13:308-311 (2001); Muñoz et al., Chem. Mater 15:500-503 (2003); and Horcajada et al., Microporous Mesoporous Mater 68:105-109 (2004).

Combination of antibodies and nanoparticles. Nanoparticles were surface modified with 2-carboxyethyl phosphonic acid to form particles with —COOH groups as surface-pendant groups. These carboxyl group functionalized particles were coupled with antibody through amidation between carboxylic (—COOH) acid groups of nanoparticle and amine (—NH2) group of antibody through 2,2-(ethylenedioxy) bis-ethylamine, a hydrophilic linker using carbodiimide. The modification of nano-particles and antibody and the conjugation are described in Mohapatra et al., Nanotechnology 18:385102 (2007).

### Example 2

### SiO₂ Nanoparticles Loaded with an Anti-Neoplastic Agent Kill Glioma Cells In Vitro

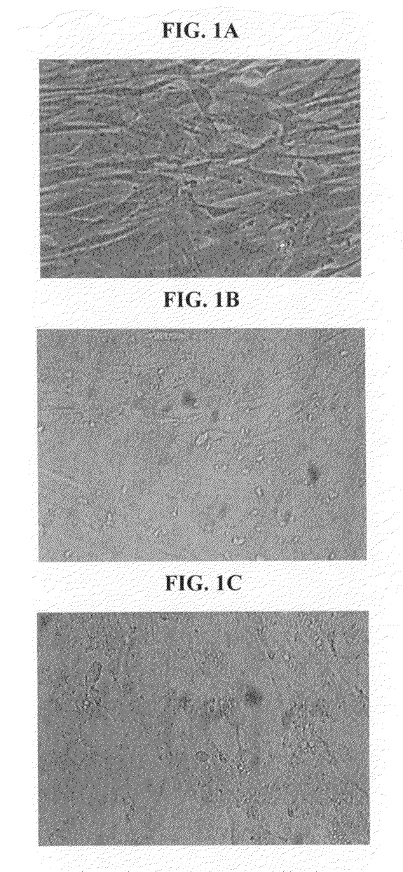

Silicate (SiO₂) nanoparticles, which were labeled with a CD90 antibody and loaded with adriamycin, were prepared as described in Example 1. This material was tested for its effects on C6 glioma cells in vitro. The anti-CD90 labeled SiO₂ nanoparticles loaded with adriamycin were added to a cell culture medium for 24 hours. C6 tumor cells were then added to the culture medium. The condition of the cells was monitored under microscopic visualization after 24 and 48 hours. The cells initially appeared normal (FIG. 1A), but the cells increasingly lost viability from 24 h (FIG. 1B) to 48 hours (FIG. 1C). As such, the anti-CD90 labeled SiO₂ nanoparticles loaded with adriamycin were effective at killing glioma cells in vitro.

A sulforhodamine B (SRB) protein assay was used to estimate cell viability or growth. The SRB test showed that the cell viability of C6 cells at 48 hours is nearly 20% of the control group contacted with the SiO₂ nanoparticles loaded with an anti-neoplastic agent. These results indicate that the SiO₂ nanoparticles loaded with an anti-neoplastic agent are useful in methods for treating glioma, when those nanoparticles are brought into contact with the glioma cells.

### Example 3

### Targeting Stem Cells with CD90-Labeled SiO₂ Nanoparticles

Anti-CD90 and FITC were combined with SiO₂ nanoparticles so that the nanoparticles could be visualized under a fluorescent microscope. The anti-CD90-FITC-SiO₂ nanoparticles were washed and filtered using a 0.22 μm filter. The SiO₂ nanoparticles are 250-300 nm in diameter and are captured by the filter.

Isolation of Human MSCs. Bone marrow samples were collected from healthy human donors (age 28-46 years). Human MSCs were isolated and cultured according to previously reported methods (Haynesworth et al., Bone, 13:81-87 (1992); Lennon et al. In Vitro Cell Dev Biol, 32:602-607 (1996); Pittenger et al. Mol Biol Cell, 7:305-309 (1996)). Briefly, cells from bone marrow aspirates were collected in heparin and fractionated over a 1.073 g/ml Percoll solution (Pharmacia Biotech, Piscataway, N.J.). Next, mononuclear cells were collected at the interface and were plated in Dulbecco's Modified Eagles Medium-Low Glucose (DMEM-LG) (Invitrogen, Grand Island, N.Y.) at a density of 3×10⁷ cells per 185 cm² flasks. The human MSC medium consisted of DMEM supplemented with 10% fetal bovine serum (FBS). Fibroblast-like cells attached and grew, developing visible symmetric colonies at about 5-7 days after initial plating. Loosely attached or nonadherent cells, including those of the hematopoietic lineage present initially in the isolated Percoll fraction, were washed away during subsequent medium changes. When the cultures reached 90% confluency, cells were subcultured using trypsin-EDTA (Life Technologies) and replated at a density of 1×10⁶ cells per 185-cm² flask. These cells expanded and remained as a monolayer and were routinely subcultured at 11-14 days.

Loading stem cells with antibody nano-particles. The nano particles, which are a black fine powder before use, were sterilized by ultraviolet light or 75% ethanol. Next, the particles were put into cell culture medium and vortexed. Insoluble materials were filtered using a 20 μm filter. The human bone marrow-derived MSCs were plated in 25 cm² flasks at 1-2×10⁴/cm². When the cells were approximately 90% confluent, they were cultured in the medium with the nano-particles for 24 hours. The cells were washed with HBSS or D-PBS 3-5 times to remove the remnant nano-particles which did not bind to the stem cells.

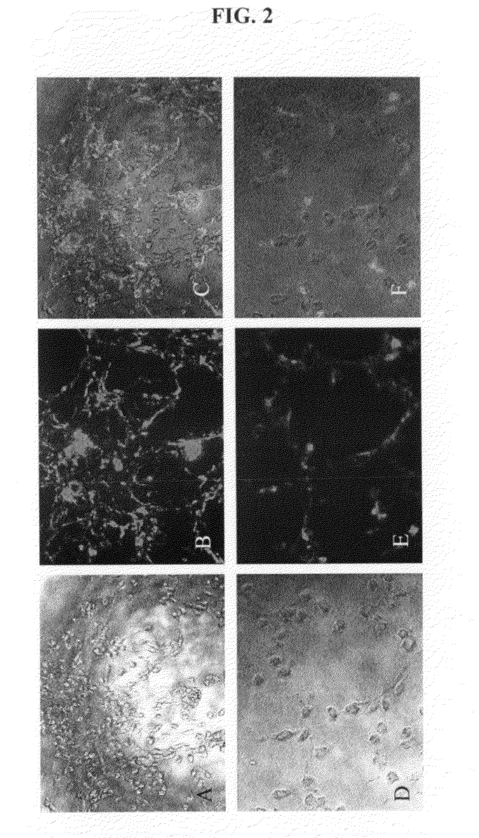

The MSCs that were contacted with anti-CD90-FITC-SiO₂ nanoparticles as described above and imaged using fluorescence microscopy. The results are shown in FIG. 2 FIG. 2A and FIG. 2D show low and high power phase contrast of MSC, respectively. FIGS. 2B and 2E show FITC fluorescence of the cells in panels A and D. FIG. 2C shows a merged image of panels A and B. FIG. 2F shows a merged image of panels D and E. These results indicate that the anti-CD90-FITC-SiO₂ nanoparticles were effectively taken up by MSCs in vitro.

### Example 4

### Preparation of Modified Stem Cells for Transplantation

Nanoparticle loaded stem cells are collected at passage 3 and 6 after being cultured as described in Example 3. They are disassociated with trypsin and treated with a trypsin inhibitor to stop the digestion. The cells are collected and resuspended to a density of 100,000 cells/μl in transplantation media (Leibowitz, L-15; Sigma), 2% B27 supplement, in 0.6% glucose in sterile PBS. The cells remain at 4° C. for the duration of the transplantation procedure.

### Example 5

### Treating Glioma in a Primate Model Using Modified Stem Cells

In this example, the modified stem cells are used to treat a glioma (e.g., cancer) in an animal model of the disease. In one instance, the modified stem cells carrying a controlled-release vehicle with an anti-neoplastic agent are targeted to glioma, known or suspected to be present in a an animal subject's body. The modified stem cells are administered parenterally or injected directly into the cranium of a subject near the site of the glioma. For example, the primate subjects may receive injections of stem cells carrying nano-particles, according to previously described protocols (Kordower et al. Science 290:767-773 (2000)).

The anti-neoplastic agent is released from the controlled release vehicle and contacted with one or more glioma cells, thereby damaging or destroying the cells. The efficacy of treatment is assessed by reduction in the number of neoplastic cells or absence of the neoplastic cells; reduction in the tumor size; inhibition (i.e., slow to some extent and preferably stop) of tumor metastasis; inhibition, to some extent, of tumor growth; increase in length of remission, and/or relief to some extent, one or more of the symptoms associated with the cancer.

### Example 6

### Treating Glioma in a Human Subject Using Modified Stem Cells

In this example, the modified stem cells are used to treat a glioma (e.g., cancer). In one instance, the modified stem cells carrying a controlled-release vehicle with an anti-neoplastic agent are targeted to glioma, known or suspected to be present in a subject's body. The modified stem cells may be administered parenterally or injected directly into the cranium of a subject near the site of the glioma.

The anti-neoplastic agent is released from the controlled release vehicle and contacted with one or more glioma cells, thereby damaging or destroying the cells. The efficacy of treatment is assessed by reduction in the number of neoplastic cells or absence of the neoplastic cells; reduction in the tumor size; inhibition (i.e., slow to some extent and preferably stop) of tumor metastasis; inhibition, to some extent, of tumor growth; increase in length of remission, and/or relief to some extent, one or more of the symptoms associated with the cancer.

### EQUIVALENTS

The present disclosure is not to be limited in terms of the particular embodiments described in this application, which are intended as single illustrations of individual aspects of. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A modified stem cell comprising a stem cell and at least one controlled-release vehicle, wherein the at least one controlled-release vehicle includes at least one anti-neoplastic agent and at least one targeting moiety, and wherein the modified stem cell is characterized by an ability to target one or more glioma cells.

2. The modified stem cell of claim 1, wherein the at least one controlled-release vehicle is selected from the group consisting of: nanoparticles; biocompatible polymers; polymeric matrices; liposomes; and lipospheres.

3. The modified stem cell of claim 1, wherein the at least one controlled-release vehicle comprises at least one nanoparticle.

4. The modified stem cell of claim 3, wherein the nanoparticle has a diameter of about 10 to about 600 nm.

5. The modified stem cell of claim 3, wherein the nanoparticle is a silicate nanoshell.

6. The modified stem cell of claim 5, wherein the silicate nanoshell is loaded with the at least one anti-neoplastic agent.

7. The modified stem cell of claim 1, wherein the controlled-release vehicle has a controlled release rate.

8. The modified stem cell of claim 7, wherein the controlled release rate is from about 5 days to about 31 days.

9. The modified stem cell of claim 1, wherein the stem cell is selected from the group consisting of: mesenchymal stem cells; neural stem cells; and embryonic stem cells.

10. The modified stem cell of claim 1, wherein the at least one targeting moiety is conjugated to the surface of the controlled-release vehicle.

11. The modified stem cell of claim 1, wherein the at least one targeting moiety is an antibody, or fragment thereof.

12. The modified stem cell of claim 11, wherein the antibody is a monoclonal antibody or a polyclonal antibody, or fragment thereof.

13. The modified stem cell of claim 1, wherein the at least one targeting moiety specifically binds a surface antigen on the stem cell.

14. The modified stem cell of claim 13, wherein the surface antigen is selected from the group consisting of: CD105 (SH2); CD73 (SH3/4); CD44; CD90 (Thy-1); CD71; Stro-1; CD106; and CD166.

15. The modified stem cell of claim 1, wherein the at least one anti-neoplastic agent is selected from the group consisting of: a chemotherapeutic agent; a protein-based pharmaceutical; and a nucleic acid-based pharmaceutical.

16. The modified stem cell of claim 1, wherein the at least one anti-neoplastic agent is selected from the group consisting of: asparaginase; adriamycin; alkaloids; alkylating agent; altretamine; amsacrine; anti-metabolite compound; antitumour antibiotics; azathioprine; bleomycin sulfate; busulfan; camptothecins; carboplatin; carmustine; chlorambucil; cisplatin; cladribine; cyclophosphamide; Cytarabine; Dacarbazine; dactinomycin; daunorubicin; docetaxel; doxorubicin hydrochloride; epipodophyllotoxins; epirubicin hydrochloride; estramustine sodium phosphate; etoposide; etoposide phosphate; finasteride; fludarabine phosphate; fluorouracil; gonadotropin-releasing hormone agonists (GnRH); Goserelin; hydroxyurea; idarubicin hydrochloride; ifosfamide; irinotecan; lomustine; marimastat; mechlorethamine; mechlorethamine hydrochloride; melphalan; mercaptopurine; methotrexate sodium; mitomycin; mitotane; mitoxantrone hydrochloride; oxaliplatin; paclitaxel; Podophyllotoxin; porfimer sodium; procarbazine hydrochloride; radiotherapeutics; streptozocin; suramin; tamoxifen; taxanes; taxol; teniposide terpenoids; thalidomide; Thioguanine; thiotepa; TNP 470; topoisomerase inhibitors; topotecan; tretinoin (all-trans retinoic acid); vinblastine; vinblastine sulfate; vinca alkaloid; vincristine; vincristine sulfate; Vindesine; vindesine sulfate; and vinorelbine tartrate.

17. The modified stem cell of claim 1, wherein the nanoparticle further comprises a labeling moiety.

18. A pharmaceutical composition comprising the modified stem cell of claim 1 and a pharmaceutically acceptable carrier.

19. A method for the treatment of glioma comprising administering to a subject in need thereof, a therapeutically effective amount of modified stem cells comprising a stem cell and at least one controlled-release vehicle, wherein the at least one controlled-release vehicle includes at least one anti-neoplastic agent and at least one targeting moiety, and wherein the modified stem cell is characterized by an ability to target one or more glioma cells.

20. A method for making a modified stem cell comprising: contacting a stem cell with at least one controlled-release vehicle having at least one anti-neoplastic agent and at least one targeting moiety.
